(19) United States
(12) Patent Application Publication
(10) Pub. No.: US 2025/0125016 A1
(43) Pub. Date: Apr. 17, 2025

(54) COMPUTER-READABLE RECORDING MEDIUM STORING INFORMATION PROCESSING PROGRAM, INFORMATION PROCESSING METHOD, AND INFORMATION PROCESSING APPARATUS

(71) Applicant: Fujitsu Limited, Kawasaki-shi (JP)

(72) Inventor: Eiji OHTA, Yokohama (JP)

(73) Assignee: Fujitsu Limited, Kawasaki-shi (JP)

(21) Appl. No.: 18/813,112

(22) Filed: Aug. 23, 2024

(30) Foreign Application Priority Data
Oct. 11, 2023 (JP) 2023-176127

(51) Int. Cl. G16C 10/00

(52) U.S. Cl. CPC G16C 10/00

(57) ABSTRACT

A non-transitory computer-readable recording medium stores an information processing program for causing a computer to execute a process including: executing a structure relaxation calculation in which a density functional theory calculation of repeatedly updating an electron density is executed a plurality of times such that the electron density is repeatedly updated until a first condition that represents that a variation rate of total electron energy based on the electron density updated last is equal to or lower than a first threshold value is satisfied in at least one time among the plurality of times in the structure relaxation calculation.

### CROSS-REFERENCE TO RELATED APPLICATION

This application is based upon and claims the benefit of priority of the prior Japanese Patent Application No. 2023-176127, filed on Oct. 11, 2023, the entire contents of which are incorporated herein by reference.

### FIELD

The embodiment discussed herein is related to a computer-readable recording medium storing an information processing program, an information processing method, and an information processing apparatus.

### BACKGROUND

In the related art, in the field of material development, there is a technique for calculating an electron density by a numerical analysis method. The numerical analysis method is, for example, a density functional theory using a self-consistent field method. For example, according to the density functional theory, an electron density is calculated by repeating a calculation of updating an electron density using a wave function until it is determined that the electron density converges. For example, when the number of atoms is N, the amount of calculation of calculating the electron density is O(N\hat{ }3)) according to the density functional theory. There is a structure relaxation calculation of obtaining a stable structure of a molecule in which total electron energy is minimized by utilizing the density functional theory. For example, the stable structure of the molecule is obtained by repeatedly executing the density functional theory while updating a steric structure of the molecule.

International Publication No. WO 2021/117510, Japanese Laid-open Patent Publication No. 2012-032908, and U.S. Patent Application Publication No. 2017/0097310 are disclosed as related art.

### SUMMARY

According to an aspect of the embodiments, a non-transitory computer-readable recording medium stores an information processing program for causing a computer to execute a process including: executing a structure relaxation calculation in which a density functional theory calculation of repeatedly updating an electron density is executed a plurality of times such that the electron density is repeatedly updated until a first condition that represents that a variation rate of total electron energy based on the electron density updated last is equal to or lower than a first threshold value is satisfied in at least one time among the plurality of times in the structure relaxation calculation.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is an explanatory diagram illustrating one example of an information processing method according to an embodiment;

FIG. 2 is an explanatory diagram illustrating an example of an information processing system;

FIG. 3 is a block diagram illustrating a hardware configuration example of an information processing apparatus;

FIG. 4 is a block diagram illustrating a functional configuration example of the information processing apparatus;

FIG. 5 is an explanatory diagram (part 1) illustrating an operation example of the information processing apparatus;

FIG. 6 is an explanatory diagram (part 2) illustrating the operation example of the information processing apparatus;

FIG. 7 is an explanatory diagram (part 3) illustrating the operation example of the information processing apparatus;

FIG. 8 is an explanatory diagram (part 4) illustrating the operation example of the information processing apparatus;

FIG. 9 is an explanatory diagram (part 1) illustrating a comparison result with a method in the related art;

FIG. 10 is an explanatory diagram (part 2) illustrating the comparison result with the method in the related art;

FIG. 11 is a flowchart illustrating an example of an overall process procedure;

FIG. 12 is a flowchart illustrating an example of a structure relaxation calculation process procedure; and

FIG. 13 is a flowchart illustrating an example of a density functional theory process procedure.

### DESCRIPTION OF EMBODIMENTS

For example, there is a technique of determining a stable structure of a molecule corresponding to a first local minimum point detected based on a gradient from molecular coordinates of the molecule over a potential energy curved surface of the molecule. For example, there is a technique in which a charge allocation structure obtained by allocating a charge amount to each atom by a charge equilibrium method in a schematic structure determined based on a chemical structural formula of a molecule or an ion is subjected to structural optimization by a molecular force field calculation method, and an initial structure of a structure optimization calculation of a quantum chemical calculation method is generated. For example, there is a technique in which a multilayer matrix of a multi-element crystal is determined based on layers of the multi-element crystal and a composition ratio of transition metals included in the multi-element crystal.

Meanwhile, in the related art, there is a case where an increase in processing time taken to execute the structure relaxation calculation by utilizing the density functional theory is caused. For example, in a case where the density functional theory for calculating an electron density is repeatedly executed while a steric structure of a molecule is updated in the structure relaxation calculation, the amount of calculation of calculating the electron density is increased, and as a result, a processing time for executing the structure relaxation calculation may be increased.

According to one aspect, an object of the present disclosure is to reduce a processing time taken to execute a structure relaxation calculation.

Hereinafter, an embodiment of an information processing program, an information processing method, and an information processing apparatus according to the present disclosure will be described in detail with reference to the drawings.

### (Example of Information Processing Method According to Embodiment)

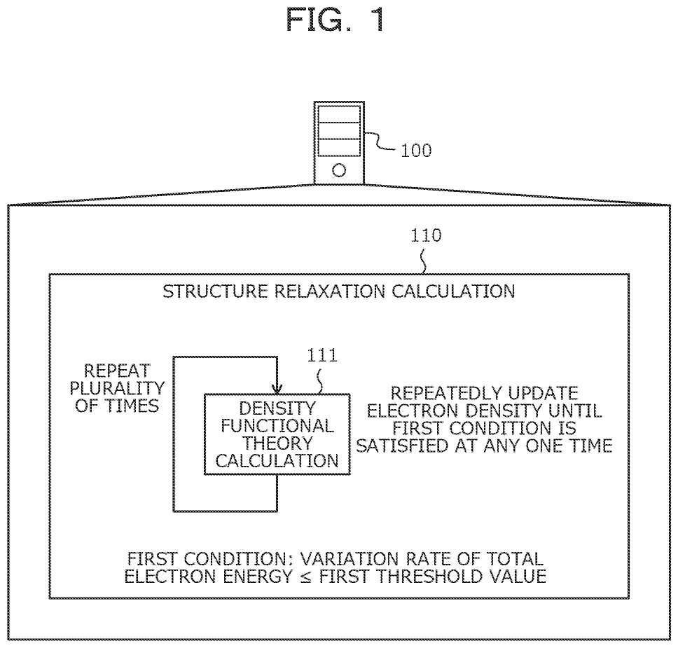

FIG. 1 is an explanatory diagram illustrating one example of an information processing method according to the embodiment. An information processing apparatus 100 is a computer for reducing a processing time taken to execute a structure relaxation calculation. For example, the information processing apparatus 100 is a server, a personal computer (PC), or the like.

The structure relaxation calculation is, for example, a series of processes for obtaining a stable structure of a molecule. For example, the structure relaxation calculation corresponds to an iterative solution method. The iterative solution method is a method in which a specific calculation is repeatedly executed until it is determined that a solution converges. The convergence is, for example, a state in which the calculated solution is equal to or lower than a threshold value. For example, the structure relaxation calculation obtains a stable structure of a molecule by executing a density functional theory for calculating an electron density and repeatedly executing a series of processes for calculating a magnitude of a force applied to an atom based on the electron density while updating a steric structure of the molecule until a convergence condition is satisfied. The convergence condition is a condition for determining that a solution converges. The convergence condition is, for example, that the magnitude of the force applied to the atom calculated last is equal to or lower than a threshold value.

The density functional theory is, for example, a series of processes for calculating an electron density. The density functional theory corresponds to, for example, an iterative solution method. According to the density functional theory, for example, the electron density is calculated by repeatedly executing a process of updating the electron density until the convergence condition is satisfied. The density functional theory corresponds to, for example, a self-consistent field method. According to the density functional theory, for example, a wave function is used to repeatedly execute a series of processes of updating the electron density at each point in a space until the convergence condition is satisfied, thereby calculating the electron density at each point in the space. The convergence condition is a condition for determining that a solution converges. The convergence condition is, for example, that a difference between an electron density calculated last and an electron density calculated immediately before becomes equal to or lower than a threshold value.

For example, it is considered that the structure relaxation calculation is used to obtain a stable structure of a molecule that controls properties of a substance in the field of material development. For example, in the field of material development, the structure relaxation calculation is used to obtain a stable structure of a molecule, and accumulate useful material data for material informatics for shortening a study period, reducing a study cost, and the like. For example, Reference Document 1 below may be referred to for the structure relaxation calculation and the density functional theory.

Reference Document 1: “Density Functional Theory: A Practical Introduction”, Author: D. S. Sholl, J. A. Steckel, translated by Taizo Sasaki, Shigeru Suehara, Publisher: Yoshioka Shoten, ISBN978-4842703657

Depending on a problem to be calculated, it may take a long time to determine that a solution converges in the density functional theory. For example, in the structure relaxation calculation, when the density functional theory is executed for the first time, there is a case where it takes a long time to determine that a solution converges in the density functional theory. For example, in the density functional theory executed for the first time in the structure relaxation calculation, the number of times a series of processes of updating an electron density at each point in a space is repeated may be increased. Therefore, in some cases, an increase in processing time taken to execute the density functional theory for the first time is caused, and an increase in processing time taken to execute the structure relaxation calculation is caused.

For example, in a case where the density functional theory is executed for the first time, when the total electron energy is shifted to a state of minute fluctuations as a result of a predetermined condition being satisfied, this leads to an increase in the number of repetitions of a series of processes of updating the electron density at each point in the space. The predetermined condition is that atoms located at relatively greatly different positions exist in an initial structure of a molecule and a stable structure of the molecule. For example, the predetermined condition is that a point at which a variation rate of a difference between an electron density calculated last and an electron density calculated immediately before is relatively small exists in the space, and it is difficult for the electron density at the point to converge.

Accordingly, in the present embodiment, an information processing method capable of reducing a processing time taken to execute a structure relaxation calculation by utilizing a density functional theory will be described. Hereinafter, the density functional theory may be referred to as a “density functional theory (DFT) method”.

As illustrated in FIG. 1, the information processing apparatus 100 stores a convergence condition related to a structure relaxation calculation 110. The convergence condition is, for example, that a calculated magnitude of a force applied to an atom is equal to or lower than a threshold value. The information processing apparatus 100 stores a first condition that may be a convergence condition related to a density functional theory. The first condition is, for example, that a variation rate of total electron energy based on an electron density updated last is equal to or lower than a first threshold value. Besides the first condition, the information processing apparatus 100 may store a second condition that may be a convergence condition related to the density functional theory. The second condition is that a difference between an electron density calculated last and an electron density calculated immediately before is equal to or lower than a second threshold value.
(1-1) The information processing apparatus 100 executes the structure relaxation calculation 110 in which a density functional theory calculation 111 for repeatedly updating an electron density is executed a plurality of times. For example, the information processing apparatus 100 executes the structure relaxation calculation 110 to repeatedly update the electron density until the first condition is satisfied in at least one time among the plurality of times in the structure relaxation calculation 110. The one time is, for example, the first time. For example, the information processing apparatus 100 executes the structure relaxation calculation 110 such that the electron density is repeatedly updated until the first condition is satisfied in the first time among the plurality of times in the structure relaxation calculation 110.

For example, the information processing apparatus 100 executes the structure relaxation calculation 110 such that the electron density is repeatedly updated until the first condition is satisfied in the first time among the plurality of times, and the electron density is repeatedly updated until the second condition is satisfied at each time other than the first time. For example, the information processing apparatus 100 may execute the structure relaxation calculation 110 such that the electron density is repeatedly updated until at least one of the first condition and the second condition is satisfied, in the first time among the plurality of times.

Thus, the information processing apparatus 100 may reduce a processing time taken to execute the density functional theory at least in the first time among the plurality of times. Therefore, the information processing apparatus 100 may reduce the processing time taken to execute the structure relaxation calculation 110. For example, even when the total electron energy is shifted to a state of minute fluctuations in the first time, the information processing apparatus 100 may reduce the number of times the series of processes of updating the electron density is repeated, and may reduce the processing time taken to execute the density functional theory. At each time other than the first time among the plurality of times, the information processing apparatus 100 may facilitate execution of the density functional theory with high accuracy, and may facilitate execution of the structure relaxation calculation 110 with high accuracy.

In a case where the convergence condition is satisfied in the structure relaxation calculation 110, the information processing apparatus 100 outputs a stable structure of a molecule. An output format is, for example, displaying on a display, a printing output to a printer, transmission to an external apparatus by a network I/F, storage in a storage region, or the like. The information processing apparatus 100 outputs the stable structure of the molecule such that a user may refer to the stable structure. Thus, the information processing apparatus 100 may enable the user to use the stable structure of the molecule.

Although the case where the function as the information processing apparatus 100 is implemented by a single computer is described, the embodiment is not limited to this case. For example, there may be a case where the function as the information processing apparatus 100 is implemented by cooperation of a plurality of computers. For example, there may be a case where the function as the information processing apparatus 100 is implemented over a cloud.

### (Example of Information Processing System 200)

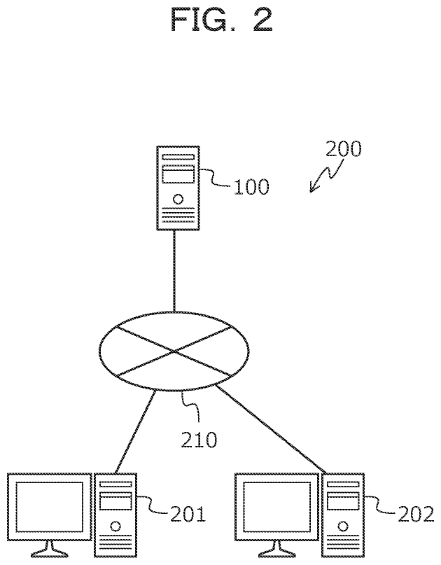

Next, an example of an information processing system 200 to which the information processing apparatus 100 illustrated in FIG. 1 is applied will be described with reference to FIG. 2.

FIG. 2 is an explanatory diagram illustrating an example of the information processing system 200. As illustrated in FIG. 2, the information processing system 200 includes the information processing apparatus 100, a numerical value calculation apparatus 201, and a client apparatus 202.

In the information processing system 200, the information processing apparatus 100 and the client apparatus 202 are coupled to each other via a wired or wireless network 210. For example, the network 210 is a local area network (LAN), a wide area network (WAN), the Internet, or the like. In the information processing system 200, the information processing apparatus 100 and the numerical value calculation apparatus 201 are coupled to each other via the wired or wireless network 210.

The information processing apparatus 100 is a computer that manages a structure relaxation calculation. For example, the information processing apparatus 100 receives a calculation instruction from the client apparatus 202. The calculation instruction designates a problem to be calculated. The information processing apparatus 100 sets a convergence condition related to the structure relaxation calculation. The convergence condition is, for example, that a calculated magnitude of a force applied to an atom is equal to or lower than a threshold value.

The information processing apparatus 100 sets a first condition that may be a convergence condition related to a density functional theory. The first condition is, for example, that a variation rate of total electron energy based on an electron density updated last is equal to or lower than a first threshold value. Besides the first condition, the information processing apparatus 100 sets a second condition that may be a convergence condition related to the density functional theory. The second condition is that a difference between an electron density calculated last and an electron density calculated immediately before is equal to or lower than a second threshold value. According to the calculation instruction, the information processing apparatus 100 executes the structure relaxation calculation based on the set convergence condition, first condition, and second condition.

For example, the information processing apparatus 100 initializes a structure of a molecule. For example, the information processing apparatus 100 executes a first density functional theory based on the first condition and the second condition with reference to the structure of the molecule. For example, the information processing apparatus 100 executes a second or subsequent density functional theory based on the second condition while updating the structure of the molecule until the convergence condition is satisfied. Thus, the information processing apparatus 100 specifies a stable structure of the molecule. According to the structure relaxation calculation, in a case where the convergence condition is satisfied, the information processing apparatus 100 transmits the stable structure of the molecule to the client apparatus 202.

For example, the information processing apparatus 100 may cause the numerical value calculation apparatus 201 to perform a distribution process on the structure relaxation calculation. The information processing apparatus 100 transmits a processing request for the structure relaxation calculation to the numerical value calculation apparatus 201. The information processing apparatus 100 may receive the stable structure of the molecule from the numerical value calculation apparatus 201, and transmit the stable structure of the molecule to the client apparatus 202. For example, the information processing apparatus 100 is a server, a PC, or the like.

The numerical value calculation apparatus 201 is a computer that shares the structural relaxation theory. According to the reception of the processing request, the numerical value calculation apparatus 201 may execute the structure relaxation calculation. The numerical value calculation apparatus 201 transmits the stable structure of the molecule to the information processing apparatus 100. For example, the numerical value calculation apparatus 201 is a server, a PC, or the like. The client apparatus 202 is a computer that transmits a calculation instruction to the information processing apparatus 100 based on an operation input by a user. After receiving the stable structure of the molecule from the information processing apparatus 100, the client apparatus 202 outputs the stable structure of the molecule to be referred to by the user. The client apparatus 202 is, for example, a PC, a tablet terminal, a smartphone, or the like. In the following description, a case where the information processing apparatus 100 operates alone will be mainly described below.

### (Application Example of Information Processing Apparatus 100)

For example, it is considered that the information processing apparatus 100 is applied to a case where a structure relaxation calculation is executed in order to obtain a stable structure of a molecule that controls properties of a substance in the field of material development. For example, it is considered that the information processing apparatus 100 is applied to a case where useful material data is accumulated by executing a structure relaxation calculation to obtain a stable structure of a molecule for material informatics.

### (Hardware Configuration Example of Information Processing Apparatus 100)

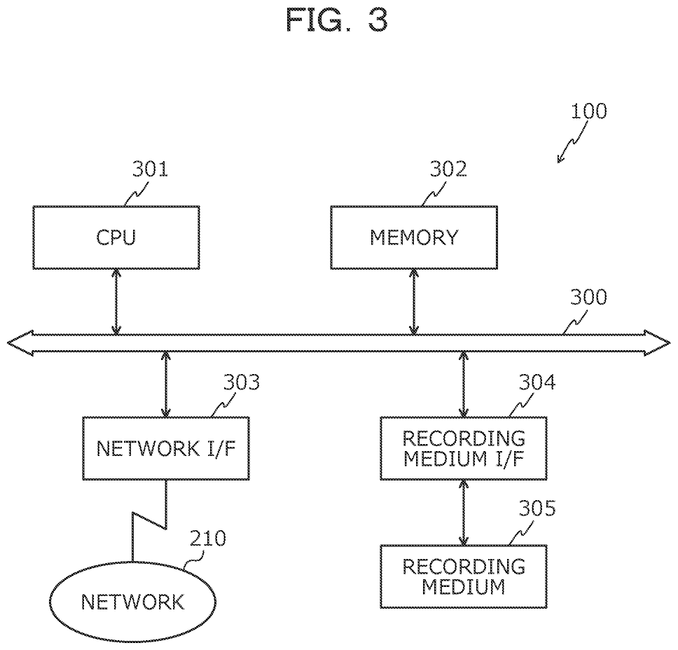

Next, a hardware configuration example of the information processing apparatus 100 will be described with reference to FIG. 3.

FIG. 3 is a block diagram illustrating the hardware configuration example of the information processing apparatus 100. In FIG. 3, the information processing apparatus 100 includes a central processing unit (CPU) 301, a memory 302, a network interface (I/F) 303, a recording medium I/F 304, and a recording medium 305. The respective components are coupled to each other by a bus 300.

The CPU 301 is responsible for control of the overall information processing apparatus 100. For example, the memory 302 includes a read-only memory (ROM), a random-access memory (RAM), a flash ROM, and the like. For example, the flash ROM and the ROM store various programs, and the RAM is used as a work area of the CPU 301. The programs stored in the memory 302 are loaded by the CPU 301, and cause the CPU 301 to execute a coded process.

The network I/F 303 is coupled to the network 210 through a communication line, and is coupled to another computer via the network 210. The network I/F 303 serves as an interface between the network 210 and the inside of the information processing apparatus 100, and controls input and output of data from and to another computer. For example, the network I/F 303 is a modem, a LAN adapter, or the like.

The recording medium I/F 304 controls reading and writing of data from and to the recording medium 305 according to the control of the CPU 301. For example, the recording medium I/F 304 is a disk drive, a solid-state drive (SSD), a Universal Serial Bus (USB) port, or the like. The recording medium 305 is a non-volatile memory that stores data written under control of the recording medium I/F 304. For example, the recording medium 305 is a disk, a semiconductor memory, a USB memory, or the like. The recording medium 305 may be removably attached to the information processing apparatus 100.

In addition to the components described above, the information processing apparatus 100 may include, for example, a keyboard, a mouse, a display, a printer, a scanner, a microphone, a speaker, and the like. The information processing apparatus 100 may include a plurality of recording media I/Fs 304 and a plurality of recording media 305. The information processing apparatus 100 may include no recording medium I/F 304 or recording medium 305.

### (Functional Configuration Example of Information Processing Apparatus 100)

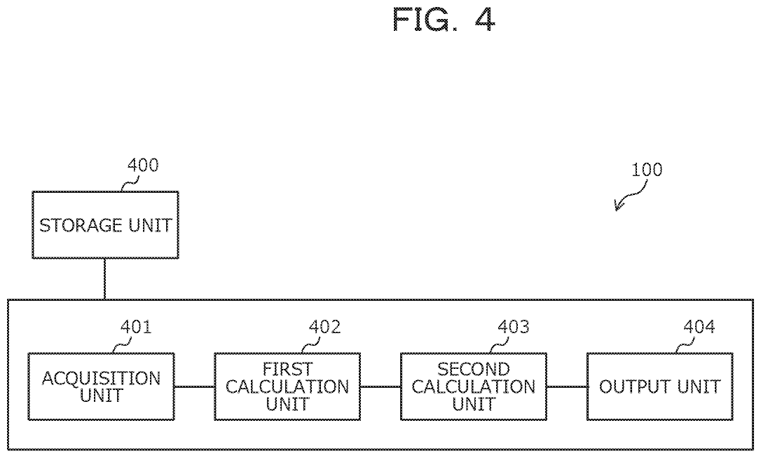

Next, a functional configuration example of the information processing apparatus 100 will be described with reference to FIG. 4.

FIG. 4 is a block diagram illustrating the functional configuration example of the information processing apparatus 100. The information processing apparatus 100 includes a storage unit 400, an acquisition unit 401, a first calculation unit 402, a second calculation unit 403, and an output unit 404.

For example, the storage unit 400 is implemented by a storage region of the memory 302, the recording medium 305, or the like illustrated in FIG. 3. Hereinafter, a case will be described where the storage unit 400 is included in the information processing apparatus 100. Meanwhile, the embodiment is not limited to this. For example, there may be a case where the storage unit 400 is included in an apparatus different from the information processing apparatus 100 and contents stored in the storage unit 400 may be referred to from the information processing apparatus 100.

The acquisition unit 401 to the output unit 404 function as an example of a control unit. For example, the functions of the acquisition unit 401 to the output unit 404 are implemented by, for example, causing the CPU 301 to execute programs stored in the storage region of the memory 302, the recording medium 305, or the like illustrated in FIG. 3 or by using the network I/F 303. For example, a processing result of each functional unit is stored in the storage region of the memory 302, the recording medium 305, or the like illustrated in FIG. 3.

The storage unit 400 stores various types of information to be referred to or updated in a process of each functional unit. For example, the storage unit 400 stores a convergence condition related to a structure relaxation calculation. The convergence condition is, for example, that a calculated magnitude of a force applied to an atom is equal to or lower than a threshold value. For example, the convergence condition is acquired by the acquisition unit 401. For example, the convergence condition may be set in advance by a user.

For example, the storage unit 400 stores a first condition that may be a convergence condition related to a density functional theory. The first condition is, for example, that a variation rate of total electron energy based on an electron density updated last is equal to or lower than a first threshold value. The electron density is, for example, an electron density at each point in a designated space. For example, the first condition is acquired by the acquisition unit 401. For example, the first condition may be set in advance by the user.

For example, the storage unit 400 stores a second condition that may be a convergence condition related to the density functional theory. The second condition is that a difference between an electron density calculated last and an electron density calculated immediately before is equal to or lower than a second threshold value. For example, the second condition is acquired by the acquisition unit 401. For example, the second condition may be set in advance by the user.

The acquisition unit 401 acquires various types of information used in a process of each functional unit. The acquisition unit 401 stores the acquired various types of information in the storage unit 400, or outputs the acquired various types of information to each functional unit. The acquisition unit 401 may output the various types of information stored in the storage unit 400, to each functional unit. For example, the acquisition unit 401 acquires the various types of information based on an operation input by the user. For example, the acquisition unit 401 may receive the various types of information from an apparatus different from the information processing apparatus 100.

For example, the acquisition unit 401 acquires a convergence condition. For example, the acquisition unit 401 acquires the convergence condition by accepting an input of the convergence condition based on an operation input of the user. For example, the acquisition unit 401 acquires the convergence condition by receiving the convergence condition from another computer.

For example, the acquisition unit 401 acquires the first condition. For example, the acquisition unit 401 acquires the first condition by accepting an input of the first condition based on an operation input of the user. For example, the acquisition unit 401 acquires the first condition by receiving the first condition from another computer.

For example, the acquisition unit 401 acquires the second condition. For example, the acquisition unit 401 acquires the second condition by accepting an input of the second condition based on an operation input of the user. For example, the acquisition unit 401 acquires the second condition by receiving the second condition from another computer.

For example, the acquisition unit 401 acquires a calculation instruction. The calculation instruction designates a problem to be calculated. For example, the acquisition unit 401 acquires the calculation instruction by accepting an input of the calculation instruction based on an operation input of the user. For example, the acquisition unit 401 acquires the calculation instruction by receiving the calculation instruction from another computer.

The acquisition unit 401 may accept a start trigger for starting a process of any of the functional units. The start trigger is, for example, a predetermined operation input by the user. The start trigger may be, for example, reception of predetermined information from another computer. For example, the start trigger may also be an output of the predetermined information from any of the functional units. As the start trigger for starting the process of the first calculation unit 402 and the second calculation unit 403, the acquisition unit 401 accepts the acquisition of the calculation instruction.

The first calculation unit 402 executes a structure relaxation calculation. For the structure relaxation calculation, a density functional theory calculation is executed a plurality of times. For example, for the structure relaxation calculation, the density functional theory calculation is executed a plurality of times by repeatedly executing the density functional theory calculation until the convergence condition is satisfied. For example, in the structure relaxation calculation, a stable structure of a molecule is obtained by executing the density functional theory calculation a plurality of times. The density functional theory calculation repeatedly updates an electron density. The plurality of times is, for example, a variation value. For example, an upper limit for the plurality of times may be set. For example, the plurality of times may be a fixed value, in some cases. For example, in the structure relaxation calculation, the first calculation unit 402 controls the second calculation unit 403 to execute the density functional theory.

For example, the first calculation unit 402 initializes a structure of a molecule by initializing an atomic position, and then repeatedly executes a series of processes. For example, the series of processes includes a process A in which the second calculation unit 403 is controlled based on the atomic position in the structure of the molecule to execute the density functional theory calculation. For example, in the process A, when the density functional theory calculation at a specific time is executed among the plurality of times, the second calculation unit 403 is controlled such that the first condition is set as a convergence condition by the second calculation unit 403. For example, the specific time is any time of the plurality of times of executing the density functional theory calculation. The specific time is, for example, the first time. For example, the specific time may be the second time or later.

For example, in the process A, when the density functional theory calculation at a specific time is executed among the plurality of times, the second calculation unit 403 may be controlled such that the convergence condition is set to satisfy at least one of the first condition and the second condition by the second calculation unit 403. For example, in the process A, when the density functional theory calculation other than the specific time is executed among the plurality of times, the second calculation unit 403 may be controlled such that the second condition is set as the convergence condition by the second calculation unit 403.

For example, in the process A, when the density functional theory calculation is executed each time before the specific time among the plurality of times, the second calculation unit 403 may be controlled such that the first condition is set as the convergence condition by the second calculation unit 403. For example, in the process A, when the density functional theory calculation is executed each time before the specific time among the plurality of times, the second calculation unit 403 may be controlled such that at least one condition of the first condition and the second condition is set to be satisfied as the convergence condition by the second calculation unit 403. For example, in the process A, when the density functional theory calculation is executed each time after the specific time among the plurality of times, the second calculation unit 403 may be controlled such that the second condition is set as the convergence condition by the second calculation unit 403.

For example, the series of processes includes a process B that is executed next to the process A and that calculates total electron energy based on an electron density at each point in a space calculated in the density functional theory calculation. For example, the series of processes includes a process C that is executed next to the process B, determines whether or not the convergence condition is satisfied, and updates the atomic position in a case where the convergence condition is not satisfied. For example, in a case where the convergence condition is not satisfied when the series of processes is executed, the first calculation unit 402 repeatedly executes the series of processes to execute the series of processes again.

Thus, the first calculation unit 402 may execute the structure relaxation calculation, and may obtain a stable structure of a molecule. At a time of executing each of a plurality of times of density functional theory calculations, the first calculation unit 402 may control the second calculation unit 403 such that the second calculation unit 403 selectively uses the first condition and the second condition as the convergence condition by the second calculation unit 403. Therefore, the first calculation unit 402 may reduce the processing time taken to execute the structure relaxation calculation, for example.

Under the control of the first calculation unit 402, the second calculation unit 403 executes the density functional theory calculation. For example, the second calculation unit 403 executes the density functional theory calculation to repeatedly update an electron density until the first condition is satisfied, at a specific time, among the plurality of times of executing the density functional theory calculation. For example, the specific time is at least one time of the plurality of times when the density functional theory calculation is executed. The specific time is, for example, the first time. For example, the specific time may be the second time or later. For example, the first condition represents that a variation rate of total electron energy based on an electron density updated last is equal to or lower than a first threshold value.

The variation rate is calculated based on a representative value of the total electron energy based on the electron density updated up to the immediately preceding time and the total electron energy based on the electron density updated last. The representative value is, for example, a statistical value. Examples of the statistical value include a maximum value, a minimum value, an average value, a mode value, a median value, or the like. For example, the representative value may be any one of the total electron energy based on the electron density updated up to the immediately preceding time. For example, the variation rate is an absolute value of a ratio of a difference between the representative value of the total electron energy based on the electron density updated up to the immediately preceding time and the total electron energy based on the electron density updated last to the representative value of the total electron energy based on the electron density updated up to the immediately preceding time. Thus, the second calculation unit 403 may reduce a processing time taken to execute the density functional theory calculation at the specific time, and may easily reduce a processing time taken to execute the structure relaxation calculation.

For example, the second calculation unit 403 may execute the density functional theory calculation such that the electron density is repeatedly updated until at least one of the first condition and the second condition is satisfied in at least a specific time, among the plurality of times of executing the density functional theory calculation. The second condition represents that, for example, a difference between the electron density updated last and the electron density updated immediately before is equal to or lower than a second threshold value. Thus, the second calculation unit 403 may reduce a processing time taken to execute the density functional theory calculation at the specific time, and may easily reduce a processing time taken to execute the structure relaxation calculation.

For example, the second calculation unit 403 may execute the density functional theory calculation such that the electron density is repeatedly updated until the second condition is satisfied at each time other than the specific time, among the plurality of times of executing the density functional theory calculation. Thus, the second calculation unit 403 may easily update the electron density with high accuracy when the density functional theory calculation other than the specific time is executed, and may easily obtain a stable structure of a molecule with high accuracy when the structure relaxation calculation is executed.

For example, the second calculation unit 403 may execute the density functional theory calculation such that the electron density is repeatedly updated at each time before the specific time until at least one of the first condition and the second condition is satisfied. Thus, the second calculation unit 403 may reduce a processing time taken to execute the density functional theory calculation each time before the specific time, and may easily reduce a processing time taken to execute the structure relaxation calculation.

For example, the second calculation unit 403 may execute the density functional theory calculation such that the electron density is repeatedly updated until the second condition is satisfied at each time after the specific time. Thus, the second calculation unit 403 may easily update the electron density with high accuracy when the density functional theory calculation at each time after the specific time is executed, and may easily obtain a stable structure of a molecule with high accuracy when the structure relaxation calculation is executed.

For example, after setting the electron density to an initial state, the second calculation unit 403 repeatedly executes a series of processes to update the electron density and calculate a final electron density. For example, the series of processes includes a process a of calculating and updating the electron density by using the one-electron wave function derived by solving the Kohn-Sham equation based on the current electron density. For example, the series of processes includes a process b that is executed next to the process a and that calculates a difference between the electron density immediately before the update and the electron density immediately after the update. For example, the series of processes includes a process c that is executed next to the process b, that calculates total electron energy as a physical characteristic value based on the electron density immediately after the update, and that calculates a variation rate of the total electron energy.

For example, in a case where the convergence condition is not satisfied when the series of processes is executed, the second calculation unit 403 repeatedly executes the series of processes to execute the series of processes again. For example, the convergence condition is a first condition in the density functional theory calculation at a specific time. For example, the first condition corresponds to a case where the calculated variation rate of the total electron energy is equal to or lower than the first threshold value.

For example, the convergence condition may be that at least one of the first condition and the second condition is satisfied in the density functional theory calculation at the specific time. The second condition is, for example, that a difference between an electron density calculated last and an electron density calculated immediately before is equal to or lower than the second threshold value. The electron density calculated immediately before is, for example, an electron density immediately before being updated. The electron density calculated last is, for example, an electron density immediately after the update. For example, the second condition corresponds to a case where a difference between the electron density immediately before the update and the electron density immediately after the update is equal to or lower than the second threshold value. For example, the convergence condition is the second condition in the density functional theory calculation other than the specific time.

For example, the convergence condition may be the first condition in the density functional theory calculation of each time before the specific time. For example, the convergence condition may be that at least one of the first condition and the second condition is satisfied, in the density functional theory calculation at each time before the specific time. For example, the convergence condition may be the second condition, in the density functional theory calculation at each time after the specific time. Thus, the second calculation unit 403 may easily reduce a processing time taken to execute the structure relaxation calculation, and may easily obtain a stable structure of a molecule with high accuracy.

The output unit 404 outputs a processing result of at least one of the functional units. An output format is, for example, displaying on a display, a printing output to a printer, transmission to an external apparatus through the network I/F 303, or storage in a storage region such as the memory 302 or the recording medium 305. Thus, the output unit 404 enables the user to be notified of the processing result of at least one of the functional units, thereby improving convenience of the information processing apparatus 100.

For example, the output unit 404 outputs a result of the structure relaxation calculation executed by the first calculation unit 402 such that the user may refer to the result. For example, the output unit 404 may transmit a result of the structure relaxation calculation executed by the first calculation unit 402 to another computer. Thus, the output unit 404 may enable an outside to refer to a result obtained by executing the structure relaxation calculation in the first calculation unit 402.

For example, the output unit 404 outputs the stable structure of the molecule obtained as a result of executing the structure relaxation calculation in the first calculation unit 402 such that the user may refer to the stable structure of the molecule. For example, the output unit 404 outputs the stable structure of the molecule obtained as a result of executing the structure relaxation calculation in the first calculation unit 402 such that the user may refer to the stable structure of the molecule. Thus, the output unit 404 may enable the outside to refer to the stable structure of the molecule.

For example, the output unit 404 may output the total electron energy obtained as a result of the structure relaxation calculation executed by the first calculation unit 402 such that the user may refer to the total electron energy. For example, the output unit 404 may transmit the total electron energy obtained as a result of the structure relaxation calculation executed by the first calculation unit 402 to another computer. Thus, the output unit 404 may enable the outside to refer to the total electron energy.

For example, the output unit 404 may output the final electron density obtained as a result of executing the structure relaxation calculation by the first calculation unit 402 such that the user may refer to the final electron density. For example, the output unit 404 may transmit the final electron density obtained as a result of executing the structure relaxation calculation in the first calculation unit 402 to another computer. Thus, the output unit 404 may enable the outside to refer to the electron density.

### (Operation Example of Information Processing Apparatus 100)

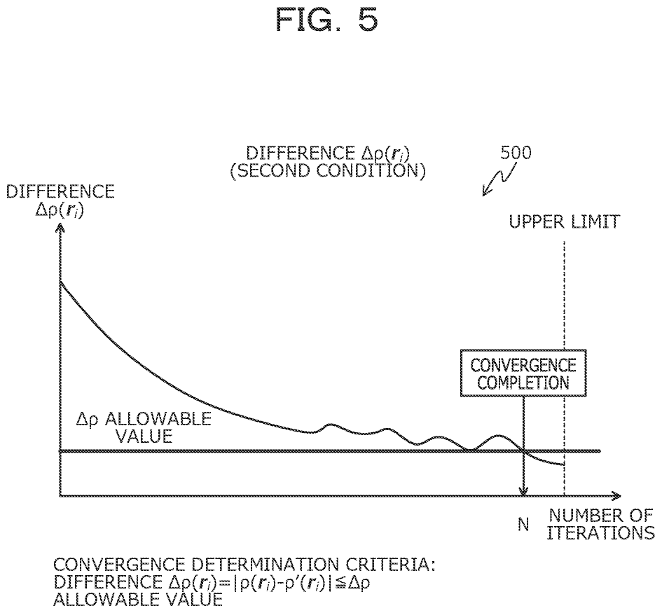

Next, an operation example of the information processing apparatus 100 will be described with reference to FIGS. 5 to 8.

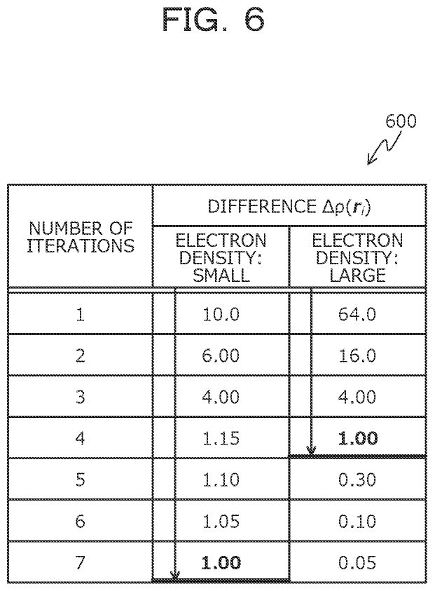

FIGS. 5 to 8 are explanatory diagrams illustrating the operation example of the information processing apparatus 100. As illustrated in FIGS. 5 and 6, the information processing apparatus 100 executes a structure relaxation calculation. In the structure relaxation calculation, when a first density functional theory calculation is executed, the information processing apparatus 100 sets that at least one of a first condition and a second condition is satisfied, as a convergence condition.

The first condition represents that a variation rate RCE of a total electron energy is equal to or lower than an allowable value RCE_TH. For example, the variation rate RCE is an absolute value of a ratio of a difference between a representative value of the total electron energy based on an electron density calculated up to the immediately preceding time and the total electron energy based on an electron density calculated last to the representative value of the total electron energy based on the electron density calculated up to the immediately preceding time. The representative value is, for example, a statistical value. Examples of the statistical value include a maximum value, a minimum value, an average value, a mode value, a median value, or the like of the electron density calculated up to the immediately preceding time.

For example, the representative value may be total electron energy based on an electron density calculated at any timing up to the immediately preceding time. For example, the representative value may be total electron energy based on an electron density calculated immediately before. For example, the allowable value RCE_TH is set in advance by a user. RCE_TH is assumed to be 1.0e-5, for example.

The second condition represents that, for all respective points rᵢ in a space, a difference absolute value Δρ(rᵢ)=|electron density ρ(rᵢ)−electron density ρ′(rᵢ)| between electron density ρ′(rᵢ) calculated last and electron density ρ(rᵢ) calculated immediately before is equal to or lower than a Δρ allowable value. For example, the Δρ allowable value is set in advance by a user. The Δρ allowable value is, for example, 1.0. By repeatedly executing a process of calculating the electron density until the convergence condition is satisfied, the information processing apparatus 100 executes the first density functional theory calculation.

The description will be continued with reference to FIGS. 5 and 6, and a timing at which the second condition is satisfied will be described. A graph 500 in FIG. 5 illustrates a change of Δρ(rᵢ) in a case where a process of calculating an electron density is repeated, and illustrates a timing at which the second condition is satisfied. A horizontal axis of the graph 500 indicates, for example, the number of iterations. The number of iterations is the number of times the process of calculating the electron density is repeated. A vertical axis of the graph 500 represents Δρ(rᵢ). As illustrated in the graph 500, the number of times the process of calculating the electron density is repeated until the second condition is satisfied is N. N is relatively close to an upper limit of the number of times the process of calculating the electron density is repeated.

A table 600 in FIG. 6 indicates a specific value of Δρ(rᵢ) in a case where the process of calculating the electron density is repeated, and indicates a timing at which the second condition is satisfied. As illustrated in the table 600, Δρ(rᵢ) corresponding to a point rᵢ at which the electron density ρ(rᵢ) is relatively small is difficult to be lower than the Δρ allowable value, as compared with Δρ(rᵢ) corresponding to a point rᵢ at which the electron density ρ(rᵢ) is relatively large. Accordingly, as illustrated in the table 600, the number N of times the process of calculating the electron density is repeated until the second condition is satisfied tends to be a relatively large number, and is, for example, 7.

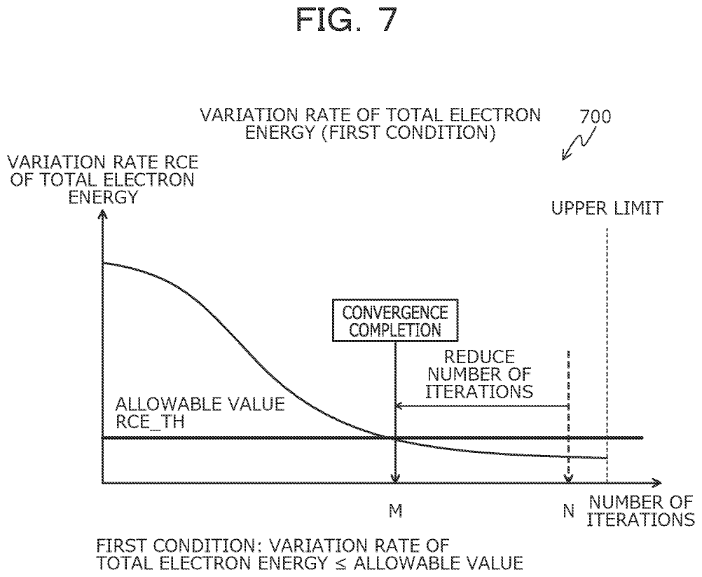

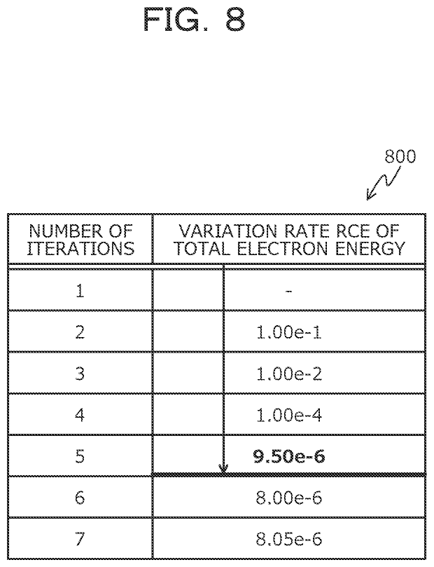

Next, the description will be continued with reference to FIGS. 7 and 8, and a timing at which the first condition is satisfied will be described. A graph 700 in FIG. 7 illustrates a change of RCE in a case where a process of calculating an electron density is repeated, and illustrates a timing at which a first condition is satisfied. A horizontal axis of the graph 700 indicates, for example, the number of iterations. The number of iterations is the number of times the process of calculating the electron density is repeated. A vertical axis of the graph 700 represents RCE. As illustrated in the graph 700, the number of times the process of calculating the electron density is repeated until the first condition is satisfied is M. M is equal to or lower than N, and is relatively far from an upper limit of the number of times the process of calculating the electron density is repeated.

A table 800 in FIG. 8 indicates a specific value of RCE in a case where the process of calculating the electron density is repeated, and indicates timings at which the first condition is satisfied. As illustrated in the table 800, RCE is likely to be lower than the allowable value RCE_TH. Accordingly, as illustrated in the table 800, the number M of times the process of calculating the electron density is repeated until the first condition is satisfied is, for example, 5. M is a value lower than N.

Thus, since the information processing apparatus 100 sets that at least one of the first condition and the second condition is satisfied as a convergence condition, it is possible to reduce the number of times the process of calculating the electron density is repeated when the first density functional theory calculation is executed. For example, the information processing apparatus 100 may reduce the number of times the process of calculating the electron density is repeated up to M times, instead of N times. Therefore, the information processing apparatus 100 may reduce a processing time taken to execute the first density functional theory calculation.

For example, a method in the related art in which only a second condition is used as a convergence condition without using a first condition is conceivable. According to this method in the related art, the number of times a process of calculating an electron density is repeated is N times. Therefore, the method in the related art leads to an increase in a processing time taken to execute a first density functional theory calculation. As compared with the method in the related art, the information processing apparatus 100 may reduce a processing time taken to execute a first density functional theory calculation, and may reduce a processing time taken to execute a structure relaxation calculation.

For the structure relaxation calculation, it is preferable that the information processing apparatus 100 sets the second condition as the convergence condition when executing the second or subsequent density functional theory calculation. Thus, since the information processing apparatus 100 does not set the first condition as the convergence condition, it is possible to easily calculate the electron density with high accuracy when the second or subsequent density functional theory calculation is executed. Accordingly, the information processing apparatus 100 may obtain a stable structure of a molecule with high accuracy when the structure relaxation calculation is executed.

### (Effect by Information Processing Apparatus 100)

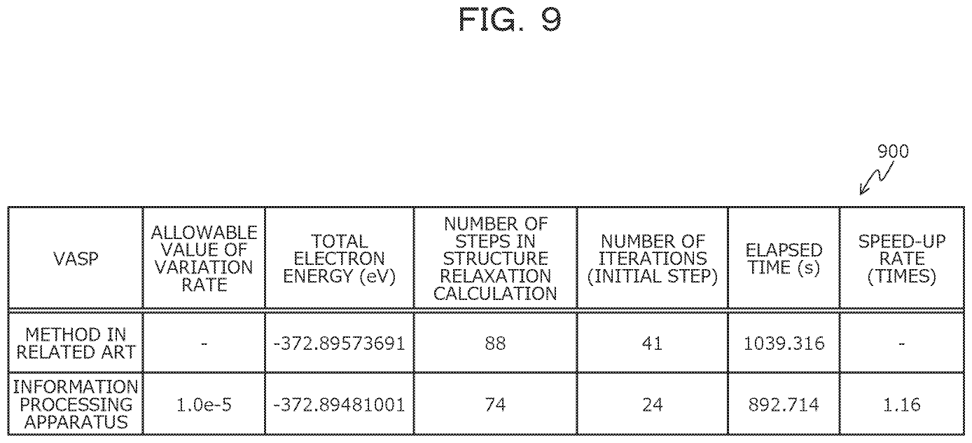

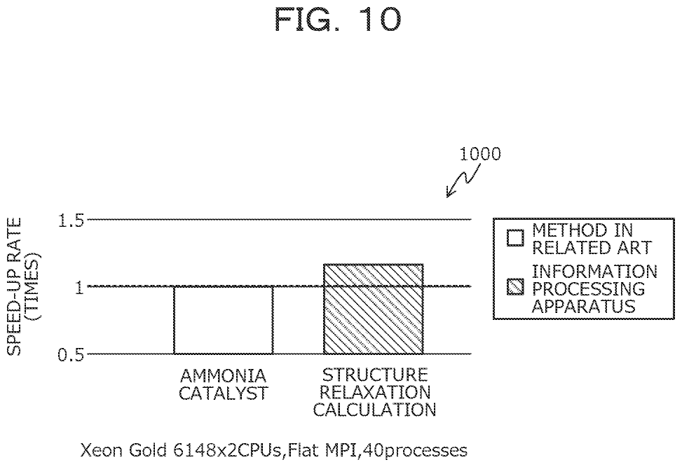

Next, an example of effects produced by the information processing apparatus 100 will be described with reference to FIGS. 9 and 10.

FIGS. 9 and 10 are explanatory diagrams illustrating a result of comparison with a method in the related art. As illustrated in FIGS. 9 and 10, the information processing apparatus 100 uses Vienna Ab initio Simulation Package (VASP) software to execute a structure relaxation calculation. The VASP software is an ab initio quantum molecular dynamics calculation program using a pseudo-potential and a plane wave basis. For example, it is assumed that the information processing apparatus 100 executes the structure relaxation calculation for the ammonia catalyst.

A table 900 in FIG. 9 illustrates a comparative example of a calculation result between the information processing apparatus 100 and a method in the related art. For example, the method in the related art uses only a second condition as a convergence condition without using a first condition. As illustrated in the table 900, the method in the related art calculates total electron energy=−372.89573691 eV. According to the method in the related art, the number of steps when a structure relaxation calculation is executed is 88 steps. According to the method in the related art, the number of iterations is 41 in a first density functional theory calculation. The number of iterations is the number of times the process of calculating an electron density is repeated. According to the method in the related art, an elapsed time when the structure relaxation calculation is executed is 1039.316 (seconds).

On the other hand, the information processing apparatus 100 sets the allowable value RCE_TH=1.0e-5. The information processing apparatus 100 calculates total electron energy=−372.89481001 eV. The number of steps when the structure relaxation calculation is executed in the information processing apparatus 100 is 74 steps. In the information processing apparatus 100, the number of iterations is 24 in the first density functional theory calculation. The number of iterations is the number of times the process of calculating the electron density is repeated. An elapsed time when the information processing apparatus 100 executes the structure relaxation calculation is 892.714 (seconds). As described above, the information processing apparatus 100 may speed up the structure relaxation calculation by 1.16 times as compared with the method in the related art.

A graph 1000 in FIG. 10 illustrates a comparative example of a speed-up rate between the information processing apparatus 100 and the method in the related art. As illustrated in FIG. 10, the speed-up rate of the information processing apparatus 100 in a case where the method in the related art is set as a criterion: 1 is 1.16 times. As compared with the method in the related art, the information processing apparatus 100 may speed up the structure relaxation calculation, and reduce an increase in processing time taken to execute the structure relaxation calculation. For example, the information processing apparatus 100 may reduce the number of steps when the structure relaxation calculation is executed by approximately 16%, as compared with the method in the related art. For example, the information processing apparatus 100 may reduce the number of times the process of calculating the electron density is repeated by approximately 41% in the first density functional theory calculation, as compared with the method in the related art. For example, the information processing apparatus 100 may accurately calculate the total electron energy with a difference lower than 0.001 eV, as compared with the method in the related art.

### (Overall Process Procedure)

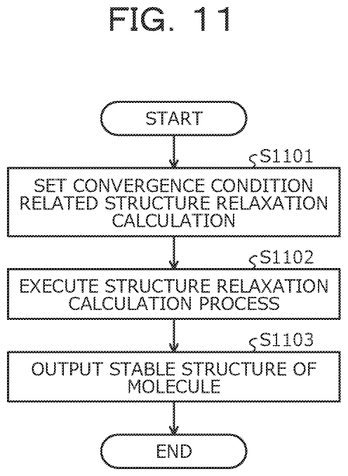

Next, an example of an overall process procedure executed by the information processing apparatus 100 will be described with reference to FIG. 11. The overall process is implemented by, for example, the CPU 301, the storage region of the memory 302, the recording medium 305, or the like, and the network I/F 303 illustrated in FIG. 3.

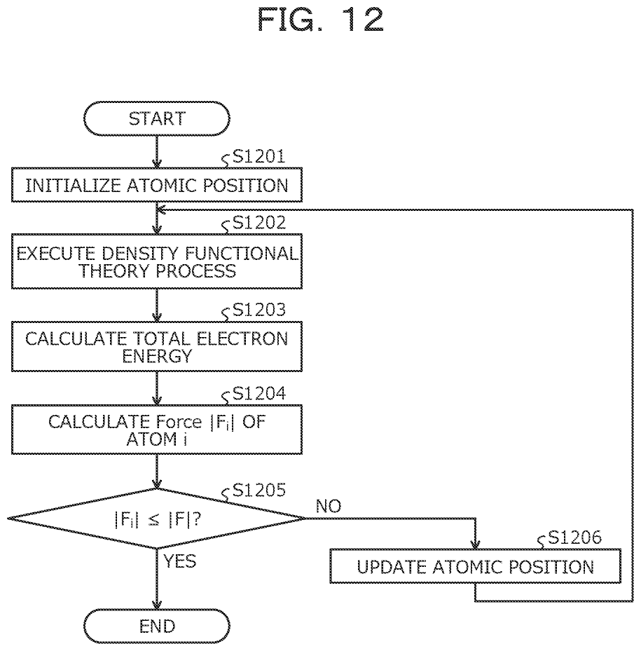

FIG. 11 is a flowchart illustrating an example of an overall process procedure. As illustrated in FIG. 11, the information processing apparatus 100 sets a convergence condition for a structure relaxation calculation (step S1101). The information processing apparatus 100 executes a structure relaxation calculation process to be described below in FIG. 12 (step S1102). The information processing apparatus 100 outputs a stable structure of a molecule (step S1103). The information processing apparatus 100 ends the overall process. Thus, the information processing apparatus 100 may enable a user to refer to the stable structure of the molecule.

### (Structure Relaxation Calculation Process Procedure)

Next, an example of a structure relaxation calculation process procedure executed by the information processing apparatus 100 will be described with reference to FIG. 12. The structure relaxation calculation process is implemented by, for example, the CPU 301, the storage regions such as the memory 302 or the recording medium 305, and the network I/F 303 illustrated in FIG. 3.

FIG. 12 is a flowchart illustrating an example of a structure relaxation calculation process procedure. As illustrated in FIG. 12, the information processing apparatus 100 initializes a structure of a molecular by initializing an atomic position (step S1201).

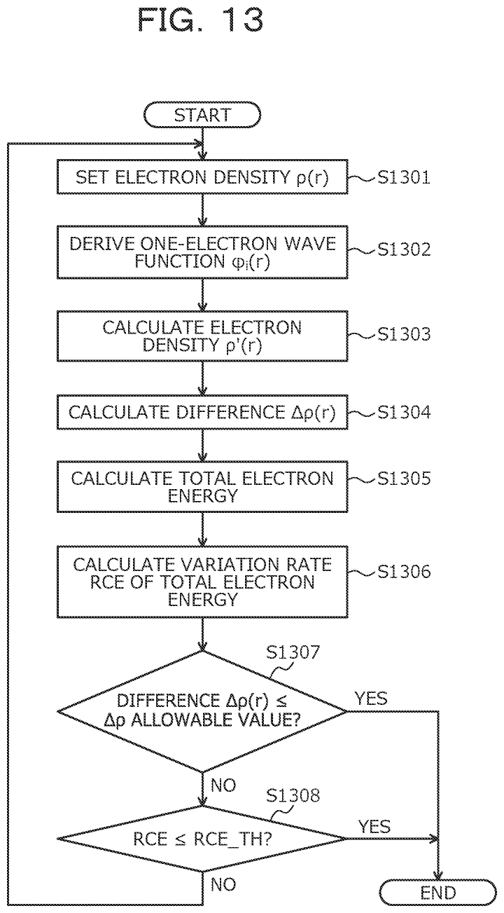

Based on the atomic position, the information processing apparatus 100 executes a density functional theory process to be described below with reference to FIG. 13 (step S1202). Based on an electron density at each point in a space, the information processing apparatus 100 calculates total electron energy (step S1203). The information processing apparatus 100 calculates Force |Fᵢ| for each atom i (step S1204).

The information processing apparatus 100 determines whether or not all of |Fᵢ|≤|F| is satisfied (step S1205). |F| is set by a user in advance, for example. In a case where all of |Fᵢ|≤|F| is not satisfied and at least one |Fᵢ|>|F| is satisfied (No in step S1205), the information processing apparatus 100 proceeds to the process in step S1206.

At step S1206, the information processing apparatus 100 updates the structure of the molecular by updating the atomic position (step S1206), and returns to the process in step S1202. On the other hand, in a case where all of |Fᵢ|≤|F| is satisfied (Yes in step S1205), the information processing apparatus 100 ends the structure relaxation calculation process. Thus, it is possible for the information processing apparatus 100 to obtain a stable structure of the molecule.

### (Density Functional Theory Process Procedure)

Next, an example of a density functional theory process procedure executed by the information processing apparatus 100 will be described with reference to FIG. 13. For example, a density functional theory process is implemented by the CPU 301 illustrated in FIG. 3, the storage region such as the memory 302 or the recording medium 305, and the network I/F 303.

FIG. 13 is a flowchart illustrating the example of the density functional theory process procedure. As illustrated in FIG. 13, the information processing apparatus 100 sets an electron density ρ(r) (step S1301). For example, in a case where there are a plurality of points in a space, the information processing apparatus 100 sets the electron density ρ(r) of each of the points. For example, in a case of a first setting, the information processing apparatus 100 sets an initial value of the electron density ρ(r). For example, the information processing apparatus 100 sets the electron density ρ(r) by any method in a case of a second or subsequent setting. For example, in a case of the second or subsequent setting, the information processing apparatus 100 sets the electron density ρ(r) to an electron density ρ′(r) calculated immediately before.

Based on the electron density ρ(r), the information processing apparatus 100 solves the Kohn-Sham equation to derive the one-electron wave function φᵢ(r) (step S1302). For example, in a case where there are the plurality of points in the space, the information processing apparatus 100 derives the one-electron wave function φᵢ(r) for each of the points. By using the one-electron wave function φᵢ(r), the information processing apparatus 100 calculates the electron density ρ′(r) (step S1303). For example, in a case where there are the plurality of points in the space, the information processing apparatus 100 calculates the electron density ρ′(r) of each of the points.

The information processing apparatus 100 calculates difference Δρ(r)=electron density ρ(r)−electron density ρ′(r) (step S1304). For example, in a case where there are the plurality of points in the space, the information processing apparatus 100 calculates the difference Δρ(r) of each of the points. Based on the electron density ρ′(r), the information processing apparatus 100 calculates total electron energy as a physical characteristic value (step S1305). The information processing apparatus 100 calculates the variation rate RCE of the total electron energy (step S1306).

The information processing apparatus 100 determines whether or not the difference Δρ(r)≤the Δρ allowable value is satisfied (step S1307). For example, in a case where there are the plurality of points in the space, the information processing apparatus 100 determines whether or not the difference Δρ(r) for all the points≤the Δρ allowable value is satisfied. In a case where the difference Δρ(r) for all the points≤the Δρ allowable value is satisfied (Yes in step S1307), the information processing apparatus 100 ends the density functional theory process. On the other hand, in a case where the difference Δρ(r) of at least one point >the Δρ allowable value is satisfied (No in step S1307), the information processing apparatus 100 proceeds to the process in step S1308.

At step S1308, the information processing apparatus 100 determines whether or not RCE≤RCE_TH is satisfied (step S1308). In a case where RCE≤RCE_TH is not satisfied (No in step S1308), the information processing apparatus 100 returns to the process in step S1301. On the other hand, in a case where RCE≤RCE_TH is satisfied (Yes in step S1308), the information processing apparatus 100 ends the density functional theory process. Thus, the information processing apparatus 100 may execute the density functional theory calculation.

For example, the density functional theory process illustrated in FIG. 13 implements the first density functional theory calculation executed by the information processing apparatus 100. For example, it is considered that the second or subsequent density functional theory calculation executed by the information processing apparatus 100 is implemented by the density functional theory process illustrated in FIG. 13 from which the process in step S1308 is deleted.

The information processing apparatus 100 may execute the processes of the steps in each flowchart illustrated in FIGS. 11 to 13 while changing a processing order of some of the steps. For example, the processing order of steps S1304 and S1305 may be changed. The information processing apparatus 100 may skip the processes of some of the steps in each flowchart illustrated in FIGS. 11 to 13.

As described above, according to the information processing apparatus 100, it is possible to store a first condition under which a variation rate of total electron energy based on an electron density updated last is equal to or lower than a first threshold value. According to the information processing apparatus 100, for a structure relaxation calculation in which a density functional theory calculation is executed a plurality of times, it is possible to execute the structure relaxation calculation such that the electron density is repeatedly updated until the first condition is satisfied in at least one time among the plurality of times. Thus, the information processing apparatus 100 may reduce a processing time taken to execute the density functional theory calculation, and reduce a processing time taken to execute the structure relaxation calculation.

According to the information processing apparatus 100, it is possible to store a second condition under which a difference between the electron density updated last and the electron density updated immediately before is equal to or lower than a second threshold value. According to the information processing apparatus 100, it is possible to execute the structure relaxation calculation such that the electron density is repeatedly updated until at least one of the first condition and the second condition is satisfied in at least one time. Thus, the information processing apparatus 100 may reduce a processing time taken to execute the density functional theory calculation, and reduce a processing time taken to execute the structure relaxation calculation.

According to the information processing apparatus 100, it is possible to repeatedly update the electron density until at least one of the first condition and the second condition is satisfied at each time before a specific time, among the plurality of times. According to the information processing apparatus 100, it is possible to repeatedly update the electron density until the second condition is satisfied at each time after the specific time, among the plurality of times. Thus, the information processing apparatus 100 may reduce a processing time taken to execute the density functional theory calculation, and reduce a processing time taken to execute the structure relaxation calculation. The information processing apparatus 100 may execute the structure relaxation calculation with high accuracy.

According to the information processing apparatus 100, it is possible to calculate the variation rate based on a representative value of the total electron energy based on the electron density updated up to the immediately preceding time and the total electron energy based on the electron density updated last. Thus, the information processing apparatus 100 may calculate the variation rate with high accuracy, may easily determine whether or not the first condition is satisfied, and may accurately determine whether or not the density functional theory calculation is ended.

According to the information processing apparatus 100, it is possible to execute the density functional theory calculation to repeatedly update the electron density by using the Kohn-Sham equation that enables calculation of the one-electron wave function. Thus, the information processing apparatus 100 may implement the density functional theory calculation.

According to the information processing apparatus 100, it is possible to execute the density functional theory calculation such that the electron density at each point in a designated space is repeatedly updated. Thus, the information processing apparatus 100 may be applied to a case where a plurality of points exist in the designated space.

According to the information processing apparatus 100, it is possible to execute the structure relaxation calculation such that the electron density is repeatedly updated until the second condition is satisfied at each time other than any one time, among the plurality of times. Thus, the information processing apparatus 100 may execute the structure relaxation calculation with high accuracy.

According to the information processing apparatus 100, it is possible to execute the structure relaxation calculation such that the electron density is repeatedly updated until the first condition is satisfied at least at a first time among the plurality of times. Thus, the information processing apparatus 100 may reduce a processing time taken to execute the density functional theory calculation, and reduce a processing time taken to execute the structure relaxation calculation. The information processing apparatus 100 may execute the structure relaxation calculation with high accuracy.

The information processing method described in the present embodiment may be implemented by causing a computer such as a PC or a workstation to execute a program prepared in advance. The information processing program described in the present embodiment is recorded in a computer-readable recording medium, and is executed by being read from the recording medium by a computer. The recording medium is a hard disk, a flexible disk, a compact disc (CD)-ROM, a magneto optical (MO) disc, a Digital Versatile Disc (DVD), or the like. The information processing program described in the present embodiment may be distributed via a network such as the Internet.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing an information processing program for causing a computer to execute a process comprising:
executing a structure relaxation calculation in which a density functional theory calculation of repeatedly updating an electron density is executed a plurality of times such that the electron density is repeatedly updated until a first condition that represents that a variation rate of total electron energy based on the electron density updated last is equal to or lower than a first threshold value is satisfied in at least one time among the plurality of times in the structure relaxation calculation.

2. The non-transitory computer-readable recording medium according to claim 1,
wherein in the executing, the structure relaxation calculation is executed such that the electron density is repeatedly updated until at least one of the first condition and a second condition that represents that a difference between the electron density updated last and the electron density updated immediately before is equal to or lower than a second threshold value is satisfied in at least the one time.

3. The non-transitory computer-readable recording medium according to claim 2,
wherein in the executing, the structure relaxation calculation is executed such that the electron density is repeatedly updated until at least one of the first condition and the second condition is satisfied at each time before a specific time among the plurality of times, and the electron density is repeatedly updated until the second condition is satisfied at each time after the specific time among the plurality of times.

4. The non-transitory computer-readable recording medium according to claim 3,
wherein the variation rate is calculated based on a representative value of total electron energy based on the electron density updated up to an immediately preceding time and total electron energy based on the electron density updated last.

5. The non-transitory computer-readable recording medium according to claim 3,
wherein the density functional theory calculation is a process of repeatedly updating the electron density by using a Kohn-Sham equation capable of calculating one-electron wave function.

6. The non-transitory computer-readable recording medium according to claim 1,
wherein the density functional theory calculation is a process of repeatedly updating an electron density at each point in a designated space.

7. The non-transitory computer-readable recording medium according to claim 1,
wherein in the executing, the structure relaxation calculation is executed such that the electron density is repeatedly updated until a second condition that represents that a difference between the electron density updated last and the electron density updated immediately before is equal to or lower than a second threshold value is satisfied at each time other than the one time among the plurality of times.

8. The non-transitory computer-readable recording medium according to claim 1,
wherein the one time is a first time.

9. An information processing method for causing a computer to execute a process comprising:
executing a structure relaxation calculation in which a density functional theory calculation of repeatedly updating an electron density is executed a plurality of times such that the electron density is repeatedly updated until a first condition that represents that a variation rate of total electron energy based on the electron density updated last is equal to or lower than a first threshold value is satisfied in at least one time among the plurality of times in the structure relaxation calculation.

10. An information processing apparatus comprising:
a memory; and
a processor coupled to the memory and configured to:
execute a structure relaxation calculation in which a density functional theory calculation of repeatedly updating an electron density is executed a plurality of times such that the electron density is repeatedly updated until a first condition that represents that a variation rate of total electron energy based on the electron density updated last is equal to or lower than a first threshold value is satisfied in at least one time among the plurality of times in the structure relaxation calculation.
